(19) United States
(12) Patent Application Publication
(10) Pub. No.: US 2007/0166389 A1
(43) Pub. Date: Jul. 19, 2007

(54) Stabilized lyophilized blood platelets

(76) Inventor: Irina B. Bakaltcheva, Springfield, VA (US)

Correspondence Address: James Remenick;Novak Druce & Quigg LLP, Suite 400 East Tower, 1300 Eye Street, N. W., Washington, DC, 20005, US

(21) Appl. No.: 11/503,371

(22) Filed: Aug. 14, 2006

Related U.S. Application Data
(60) Provisional application No. 60/707,532, filed on Aug. 12, 2005.

(51) Int. Cl. A61K 35/14; A01N 1/02; C12N 5/08

(52) U.S. Cl. 424/532; 435/2; 435/372

(57) ABSTRACT

A lyophilized platelet preparation, which is preferably a platelet enhanced plasma preparation, was obtained by stabilizing separated platelets with glyceraldehyde. In contrast to fixed preparations of cells, the platelet preparation is made and provided without toxins. Not fixing the platelets according to the invention provides increases utility and allows for the ability to change volume. The platelets are combined with a glyceraldehyde analog for a few hours at slightly elevated temperatures (about 35° C.) and then freeze dried and on reconstitution with distilled water exhibit morphological and physiological properties similar to that of native platelets, and superior to untreated, lyophilized platelets.

### REFERENCE TO RELATED APPLICATIONS

This application claims priority to U.S. Provisional Application No. 60/707,532 entitled “Stabilized, Lyophilized Blood Platelets” filed Aug. 12, 2005, the entirety of which is hereby incorporated by reference.

### RIGHTS IN THE INVENTION

This invention was made with support from the United States Government, Department of the Army, and, accordingly, the United States has certain rights in the invention.

### BACKGROUND OF THE INVENTION

1. Field of the Invention

This invention relates to lyophilized blood platelets that can be stored for long periods of time, almost indefinitely, reconstituted and subsequently administered to a patient. Platelets are not fixed prior to freezing, but stabilized with a non-toxic, glyceraldehyde analog that supports platelets and then freeze dried according to the invention. Crystallization is minimized during the process to avoid platelet damage and enhance platelet longevity.

2. Description of the Background

Lyophilized, or freeze dried platelets, have a variety of applications including use in the treatment of trauma victims where refrigeration or conventional blood banks are not generally available. One application of this type of technology is on the battlefield. Wounded soldiers, needing treatment for trauma injuries and the consequent injury due to platelet depletion and the associated reduced amount of clotting factors, could be treated with lyophilized platelets before they risk exsanguation prior to stabilization in a hospital setting. Similar applications can be found wherever human trauma injury occurs that is far from the infrastructure necessary to support platelet maintenance.

Platelets can be applied to a wounded subject using different techniques. Platelets can be applied directly upon reconstitution by placing the platelets, or platelet enriched plasma, directly on the wound to induce clotting, or in an alternative technique, through a dressing or bandage applied to the wound. These same technologies used on humans can be applied in the practice of veterinary medicine. Although many different formulations and approaches have been advanced in the last twenty years for lyophilized blood platelet preparation, the United States Food and Drug Administration (FDA) has not yet approved one for use in the medical field. One of the obstacles to delivery of an acceptable product is the preparation. Researchers previously attempted to “fix” the platelets to protect them from freeze drying, but this technique introduces both toxins and the potential for platelet damage.

Conventional liquid platelet-rich plasma concentrates are stored in blood banks at 22° C. Federal regulations require these platelets to be discarded after 5 days because of the risk of bacterial growth. This leads to an ongoing shortage of platelets in blood banks nationwide. Consequently, the development of techniques for long term cryo- or lyo-preservation of platelets has been the focus of extensive research.

Methods for platelet lyophilization storage are more versatile today because of the substantial advancements in this research field. These methods present a variety of different approaches designed to meet the high demand for lyophilized platelet products. The lyophilized products are expected to be light weight, stable at ambient temperature, and easy to ship, store and use in rural locations where no established blood bank system is in place or in emergency and disaster situations. In this regard, methods for platelet cryopreservation storage utilizing dimethyl sulfoxide have become more refined, effective, and simple to use.

The development of freeze-drying procedures for complex cellular systems such as platelets or red blood cells requires optimization of four distinct steps: pretreatment; freezing; primary drying; and secondary drying.

Pretreatment includes any method of treating the cells prior to freezing and is of special concern because employment of inadequate techniques at this phase leads to unusable platelets at reconstitution. The goal at this point in the lyophilization process is to increase cell stability against the stresses of freezing and drying. Two main approaches can be used to achieve this goal. The first approach utilizes mixtures of high and low molecular weight cryoprotectants in accordance with the principles of the glass transition theory. The second approach applies crosslinking or reversible crosslinking to assure cell stability during lyophilization.

The first approach to Pretreatment requires a low molecular weight cryoprotectant, usually a carbohydrate permeable to the cell membrane. Womersley et al. (Cryobiology, 23:245-55 (1986)) reported that in order for membrane preservation to be accomplished, the carbohydrate had to be present on both sides of the membrane and failure to achieve this would greatly diminish the capacity of the carbohydrate to protect against desiccation-induced damage (14). Carbohydrate presence on both sides of the membrane is easily achieved with phospholipid vesicles, which can be made de novo in the presence of a carbohydrate, but this procedure presents a challenge with intact cells. Recently, Wolkers et al. (Cryobiology, 41: 79-87 (2001)) have developed a method for trehalose loading into human platelets. Trehalose is a non-reducing sugar formed from two glucose units joined by a 1-1 alpha bond. In this method, platelets are heated for several hours at 37° C. in the presence of trehalose. Under these conditions, trehalose is taken up by platelets via a mechanism identified as fluid-phase endocytosis having a loading efficiency of 50% or greater. The highest internal trehalose concentration can be achieved with 52 mM external trehalose. However, to achieve successful freezing of platelets, much higher internal and external cryoprotectant concentrations are required. Trehalose loaded lyophilized human platelets have been thoroughly characterized on a molecular and a membrane level using Fourier transform infrared spectroscopy (FTIR) to depict protein secondary structure and membrane phase-transitions. However, data supporting the assumption that these platelets are fully functional on a cellular or a physiological level are very limited. Thus, trehalose loaded platelets are unsatisfactory because the reconstituted platelets must exhibit full functionality without extensive post wetting treatments to satisfy the needs of a remote or battlefield deployment of the technology.

The second approach to Pretreatment uses fixatives or crosslinkers to stabilize platelets before lyophilization. Read et al. (U.S. Pat. No 5,993,804) disclose that pharmaceutically acceptable fixed-dried platelets can be prepared by means of fixatives such as formaldehyde, paraformaldehyde, and gluteraldehyde or by using a permanganate fixate. In this procedure, platelets are preferably fixed in 1.8% paraformaldehyde and then lyophilized in the presence of 5% albumin. The hemostatic and structural properties of preparations such as the aforementioned have been well characterized during the past couple of years and long term storage of fixed, lyophilized platelets was shown to be possible. Bakaltcheva et al. (Cryobiology, 40: 343-59 (2000)) introduced a reversible crosslinking stabilization procedure for preparation of lyophilized red blood cells. In this procedure, the red blood cells are crosslinked with dimethyl 3,3-dithiobispropionimidate (DTBP) before lyophilization. The crosslinking is then reversed upon rehydration to attempt to recover some of the initial cell functionality and deformability. The above procedures apply chemical crosslinkers or fixatives known for their toxic effects, which are unacceptable for use as medicaments. Thus, a recognized major drawback of the currently available chemical crosslinking agents or fixatives used for biological tissue or cell stabilization is the toxic effects from the fixed tissue, cells or residues. Therefore, it is unlikely that, for example, paraformaldehyde or similarly fixed platelets or crosslinked/reversible crosslinked red blood cells will ever be acceptable under FDA rules and regulations, and find their place in the nation's blood banks.

Accordingly, it would be desirable to provide toxin-free, lyophilized blood platelet preparations that upon reconstitution provide full functionality.

### SUMMARY OF THE INVENTION

In accordance with an embodiment, a lyophilized preparation of blood platelets comprises, prior to lyophilization, blood platelets stabilized with glyceraldehyde, genipin, glyoxal, an analog thereof, or a combination thereof.

In an embodiment, the platelets are resuspendable in at least one of autologous plasma, allogenic plasma, a high molecular weight polymer and/or combinations thereof, prior to lyophilization.

In an embodiment, the high molecular weight polymer is at least one of a dextran, a hydroxyethyl starch, a modified gelatin, an albumin and/or combinations thereof.

In some embodiments, the autologous or allogenic plasma is a platelet poor plasma, which can further comprise at least one of a sucrose, a trehalose, a glycine, a dymethyl sulfoxide and/or combinations thereof.

In some embodiments, the said blood platelets are responsive to hypotonic stress. In some embodiments, the preparation is substantially free of toxic chemicals. In some embodiments, the lyophilized platelets are flexible.

In some embodiments, the glyceraldehyde analog is selected from the group consisting of dl-glyceraldehyde, dl-glyceraldehyde dimer, glyoxal, and combinations and mixtures thereof.

In some embodiments, subsequent to reconstitution, the platelets exhibit a size distribution and freedom from aggregation that is substantially indistinguishable from control platelets that have not been subject to lyophilization. IN some embodiments, the platelets are reconstituted in at least one of a pH adjusted matrix of autologous plasma, allogenic plasma and/or combinations thereof.

In an embodiment, the platelets are reconstituted in at least one of distilled, deionized, distilled-deionized, autoclaved, sterile saline, and ultra pure pathogen free water, and/or combinations thereof.

In some embodiments, upon reconstitution, the platelets release LDH in an amount corresponding to less than 100 U/L in a five hundred thousand cells/μl concentration.

In some embodiments, upon reconstitution, the platelets aggregate in the presence of ristocetin. In some embodiments, upon reconstitution, the platelets exhibit a hypotonic shock response of approximately 10-25% or more.

A method of preparing lyophilized platelets according to an embodiment comprises stabilizing platelets from a donor in the presence of a glyceraldehyde analog at 30-40° C. for a period of 1-3 hours, washing the platelets, suspending the platelets in a pH buffered protein matrix, and freeze drying the resuspended platelets.

In some embodiments, the method further comprises stabilizing the platelets in at least one of glyceraldehyde, genipin, glyoxal, an analog thereof, or a combination thereof.

In some embodiments, the method further comprises resuspending the platelets in a glyceraldehyde-dimer. In some embodiments, the method comprises resuspending the platelets in at least one of autologous plasma, allogenic plasma, a high molecular weight polymer and combinations thereof prior to lyophilization. In some embodiments, the method comprises reconstituting the stabilized lyophilized platelets.

Accordingly, the a lyophilized plasma preparation is configured for addressing the problems and disadvantages associated with current platelet lyophilization schemes.

Other embodiments and advantages of the invention are set forth in part in the description, which follows, and in part, may be obvious from this description, or may be learned from the practice of the invention.

### DESCRIPTION OF THE INVENTION

Conventional methods for preserving platelets involve fixing cells with paraformaldehyde, gluteraldehyde or DTBP. These chemicals preserve the cells, but the fixing, which requires complete stabilization of the platelet membrane and necessarily induces membrane damage.

It has been surprisingly discovered that glyceraldehyde and glyceraldehyde analogs can be used as a non-toxic stabilizing agents to treat, rather than fix platelets prior to lyophilization. Platelets preserved by procedures according to the instant disclosure can be stored indefinitely, can be resuspended with water, a saline-based solution and can be prepared in various forms. For purposes of the instant disclosure and claims, “water” includes, but is not limited to, at least one of distilled, deionized, distilled-deionized, autoclaved, sterile saline, ultra pure pathogen free and/or combinations thereof. Platelets preserved with procedures according to the instant disclosure can also be resuspended with at least one of autologous plasma, allogenic plasma and combinations thereof. Further, in accordance with the disclosure, the lyophilized platelets can be reconstituted in a pH adjusted matrix. The pH adjusted matrix can comprise at least one of water, plasma, autologous and/or allogenic, and/or combinations thereof.

Glyceraldehyde stabilized platelets retain some ability to change volume in response to drying, and thus, are not fixed. Glyceraldehyde and glyceraldehyde analogs are readily obtained from many chemical companies, such as for example, Sigma Inc. According to the data safety sheet, for example for glyceraldehyde, no known toxic effects are indicated.

Glyceraldehyde is an efficient antisickling agent in vitro wherein it acts primarily at the stage of aggregation of sickle cell deoxyhemoglobin. Five of the 24 amino groups per αβ dimer of hemoglobin can of form stable ketoamine linkages with a glyceraldehyde or glyceraldehyde analog in a reaction similar to the non-enzymatic glucosylation of proteins. Chromium-51 red blood cell studies, used to investigate the life span in vivo of sickle erythrocytes after glyceraldehyde treatment in vitro, have shown a prolongation of the life span of sickle erythrocytes by treatment with glyceraldehyde. Additionally, in agreement with an embodiment according to the invention, these studies failed to show any deleterious effects of the glyceraldehyde treatment.

Glyceraldehyde analogs can include dl-glyceraldehyde, dl-glyceraldehyde dimer, glyoxal, genipin and all other chemicals that behave similarly according to the invention. While genipin is chemically dissimilar from glyceraldehyde, it is a naturally occurring cross-linking reagent and reacts to stabilize the platelets in a similar manner to glyceraldehyde analogs. Genipin has been used in traditional Chinese medicine, or herbal medicine, to increase bile secretion and treat various inflammatory or hepatic diseases (Akao et al., Biol Pharm Bull, 17: 1573-76 (1994)). Moreover, it has been demonstrated in a rat model that dosages of genipin effective for increasing bile salt secretion, do not cause any abnormal symptoms in the liver or kidney and thus can be an acceptable additive to stabilize platelets (Aburada et al., Pharmacometrics, 19: 259-267 (1980)). Genipin has also been used in the immobilization of enzymes, preparation of gelatin microcapsules, bioprostheses and in the fabrication of food dyes (Fujikawa et al., Biotechnol Lett, 9: 697-702 (1987); Fujikawa et al., Tetrahedron Lett, 28: 4699-4700 (1987); Sung et al., J Biomed Mater Res, 47(2): 116-126 (1999)). Accordingly, genipin shares with glyceraldehyde an absence of toxicity in the processes and products of the invention that is dissimilar to conventional toxic fixing agents.

Additional chemicals that have the same or sufficiently similar reactivity with the platelet membrane and are non-toxic include, but are not limited to glyceraldehyde and genipin analogs, and chemicals that can be empirically identified as useful in the methods of the invention from the disclosures and teachings herein.

In one embodiment, blood is collected and platelets separated from plasma, which is then washed. The platelets can then stabilized by combination with glyceraldehyde or a glyceraldehyde analog according to the invention. Washed platelets are stabilized in two steps at 35° C. in the presence of an aldehyde with the aldehyde concentration increasing during the second step of stabilization, while the platelet count is decreased. The stabilization buffer can contain an anticoagulant to assure sufficient and effective platelet stabilization. The final washing buffer can contain a platelet disaggregation agent such as MgCl₂, MgSO₄ or Prostaglandin-E to assure recovery of the initial platelet count after completion of the stabilization process.

Stabilized platelets can be washed again, but the rigid cleaning protocols of prior art preparations used to remove formaldehyde-type materials are not needed because glyceraldehyde analogs are non-toxic. In one embodiment of the invention, stabilized platelets are resuspended in a freeze-drying matrix of autologous plasma. In another embodiment, stabilized platelets are resuspended in a freeze-drying matrix of allogenic plasma. In another embodiment, stabilized platelets are resuspended in a freeze-drying matrix of a buffered protein solution. After resuspension, stabilized platelets are dried in a controlled freezing protocol, which is designed to minimize crystallization. Protocols of this type have been developed generally for platelets and plasma. For example, Oetjen et al (Freeze-Drying, p. 323-324 (2nd Edition, 2004)) describe a staged lyophilization protocol for platelets treated with the polyol adonitol.

A support matrix is valuable for lyophilization and trehalose can be used. Also, although albumin is a common choice for addition to a suitable buffer for the platelets after stabilization, the matrix does not have to be a protein rich albumin matrix. One reason for this is because it has been shown that albumin can impair platelet hemostatic function (Bakaltcheva and Reid, Transfusion Medicine Reviews. Vol 17, No 4: 263-271(2003)).

In an embodiment of the invention, platelets are resuspended in a resuspension media of their own autologous plasma prior to freeze-drying. This type of resuspension yields additional functionality potential. Subsequent to stabilization, platelets are lyophilized. In one embodiment, lyophilization can occur in autologous platelet poor plasma supplemented with low molecular weight protectants such as, but no limited to, sucrose, trehalose, glycine, dymethyl sulfoxide or combinations thereof. In addition to using platelet poor plasma, autologous or allogenic, as a lyophilization matrix for platelets, a suitable matrix for platelet lyophilization can be designed using any high molecular weight polymer that can be used as a plasma replacement fluid. Such polymers include, but are not limited to, dextrans, hydroxyethyl starches, modified gelatins, albumin and combinations and mixtures thereof. Encompassed in the instant disclosure is resuspension in at least one of autologous plasma, allogenic plasma, high molecular weight polymers and combinations thereof

In another embodiment, a difference between “fixed-frozen” or “fixed-lyophilized” platelets and stabilized, unfixed platelets is in the difference in the reaction between the glyceraldehyde analog stabilizer and the platelets, compared to the reaction of platelets with fixatives such as gluteraldehyde. The reaction of glyceraldehyde, glyceraldehyde-dimer and glyoxal with proteins is analogous to the non-enzymatic glycation of proteins with glucose. Under physiological conditions, simple sugars participate in non-enzymatic glycation reactions in the human body. One such simple sugar is glyceraldehyde. Platelets are structurally stabilized due to binding of the permeable glycating agent to the membrane and the cytoskeleton proteins. However, according to an embodiment, platelets remain flexible. Stabilized platelets, after glyceraldehyde treatment, display an increase in Mean Platelet Volume (MPV), which indicates glyceraldehyde intake by the platelet. After lyophilization, MPV of stabilized platelets is similar to the original MPV, indicating cell shrinkage during drying. Lyophilized stabilized platelets also retain some of their original ability to respond to hypotonic stress. When placed in a hypotonic environment lyophilized stabilized platelets first swell then extrude water and contract respectively.

Lactate dehydrogenase (LDH) release data show that the addition of a lyoprotectant, such as sucrose, is necessary in order to minimize platelet damage and the associated LDH release during the process of freeze-drying and subsequent rehydration. LDH release indicates that stabilization with glyceraldehyde dimer per se does not completely solidify, fix platelets or assure complete membrane preservation. A combinatory approach using stabilizing agents along with properly selected cryo-protectants or lyoprotectants is basic to this invention.

Accordingly, the instant disclosure relates to both freeze dried platelet preparations and uses thereof. The instant disclosure also relates to a platelet enriched plasma preparations having utilities similar to those described above for the platelets themselves that can be tailored to systemic delivery.

The following examples illustrate embodiments in accordance with the invention, but should not be viewed as limiting the scope of the invention.

### EXAMPLES

A process according to an embodiment of the invention can be carried out in the following manner using the reagents glucose, NaCl, KCl, imidazole, lysine, dl-glyceraldehyde, dl-glyceraldehyde dimer, prostacyclin, ristocetin and human α-thrombin were of the highest reagent grade available and purchased from Sigma (St. Louis, Mo.). The CaCl₂ reagent used with the ST-4 and the thromboelastograph were obtained from Diagnostica Stago (Parsippany, N.J.) and Heamoscope Corporation (Niles, Ill.), respectively.

Platelet Stabilization Procedure

Stabilized-lyophilized platelets were prepared using the following non-toxic naturally occurring aldehydes: dl-glyceraldehyde, or dl-glyceraldehyde dimer. Two-Step Stabilization procedure using non-toxic aldehydes: Platelets were washed three times in Washing Buffer 1 by centrifugation (2000 g for 5 minutes) in the presence of 0.05 μg/ml prostacyclin (PGI₂), which was added to the suspension prior to each wash. The composition of Washing Buffer 1 can be: 0.4 mM NaH₂PO₄, 137 mM NaCl, 5.5 mM glucose, 2.8 mM KCL, 1.0 mM MgCl₂, 12 mM NaHCO₃ and 10 mM HEPES. After the final centrifugation wash, supernatant was removed and the platelets resuspended in Washing Buffer 1 at a final concentration of approximately 800K cells/μl. One volume of a Stabilizing Buffer 1 was then added to one volume of the platelet suspension. Stabilizing Buffer 1 contained all of the components of Washing Buffer 1 plus 2% (wt./vol) dl-glyceraldehyde and/or glyceraldehyde dimer, 2.2% (wt./vol) tri-sodium citrate, and 0.8% (wt./vol) citric acid. Platelets can be stabilized at 35° C. under gentle rotation for one hour. After one hour, one volume of the platelet suspension was then resuspended in one volume of Stabilizing Buffer 2. Stabilizing Buffer 2 contained all the components of Washing Buffer 1 plus 3% (wt./vol) dl-glyceraldehyde, 2.2% (wt./vol) tri-sodium citrate, 0.8% (wt./vol) citric acid. Platelets can then be further rotated for two hours at 35° C. After completion of the stabilization procedure the platelets were washed three times in Washing Buffer 2 by centrifugation (2000 g for 5 minutes). The composition of Washing Buffer 2 was: 145 mM NaCl, 5 mM KCl, 28 mM Imidazole, 50 mM Lysine, 2.2% (wt./vol) tri-sodiurn citrate, 0.8% (wt./vol) citric acid, 1% albumin and 10 mM MgCl₂. After the final wash, the platelet count was adjusted at 500K cells/μl using autologous platelet poor plasma (PPP), and supplemented with 60 mM sucrose and 2 mM citric acid. The stabilized platelets was then freeze-dried.

Platelet Freeze-Drying Procedure

Freeze-drying was performed using an automated FTS Kinetics lyophilizer (Stone Ridge, N.Y.). Two milliliter aliquots of platelet suspensions were transferred to plastic bottles, which were placed on the shelf of the lyophilizer at room temperature. The shelf was cooled to 5° C. at a rate of 2.5° C./min and the samples held at that temperature for 30 minutes. The shelf was further cooled to −5° C. at a rate of 2.5° C./min and the samples kept for 15 minutes at that temperature. Afterwards the samples were frozen to −35° C. at rate of 2.5° C./min, and kept for minutes at that temperature to confirm complete freezing. The absolute pressure of the lyophilization chamber was reduced to 50 mT. The shelf was then heated at a rate of 0.2° C./min to 15° C. After completion of primary drying the chamber pressure was set to 20 mT and the shelf temperature increased to 18° C. at a rate of 0.2° C./min. After the product temperature reached 18° C., samples were dried for two hours and bottles sealed with rubber stoppers (Daikyo Flourotec, West Pharmaceutical Sciences) under vacuum inside the chamber. The freeze-dried samples were stored at room temperature (22° C.). Direct rehydration was performed with water.

Rehydration

The amount of water necessary for reconstitution was determined by the following: The liquid platelet-rich plasma, pre-lyophilized, (2 ml per bottle) and the post-lyophilized platelet-rich plasma was weighed using a Mettler-Toledo balance A6104 and then averaged. The mean weight difference between the two indicated the amount of water needed for rehydration. Weight determination was performed on 20 bottles. Freeze-dried platelet-rich plasma was rehydrated using 1.8 ml distilled water per bottle. Reconstitution time was less than 2 minutes at room temperature.

Platelet Size and Volume Distribution

Platelet size and volume distributions were recorded using a cell counter (e.g., Cell-Dyne 1700, Abbott Diagnostics Division, Mountain View, Calif.).

Platelet Morphology

Platelet morphology was analyzed visually at 4 and at 16x magnification utilizing a Leitz Light Microscope (Wetzler, Germany).

Lactate Dhydrogenase (LDH) Concentration

Extracellular LDH was quantified with an automatic analyzer (e.g., Boehringer Mannheim/Hitachi 902 System). Sample processing was performed according to the manufacturers standard operating procedures as detailed in a users guide and the product inserts for the assay. All reagents and supplies are available from a chemical company such as, Roche Diagnostics.

LDH was measured in the supernatants of fresh, and lyophilized platelets, normalized for 500K cells/μl, and reported in U/L. The amount of LDH released as a result of lyophilization/rehydration was determined by subtracting the LDH amount originally present in the supernatants prior to lyophilization.

Determination of Recalcification Time

The Diagnostica Stago ST4 (Parsippany, N.J.) is a semi-automated coagulation instrument used for in vitro testing of the coagulation system employing an electromechanical clot detection system. The plasma recalcification time was carried out according to standard procedures described in hematology texts. One-third volume sample was incubated with one-third volume 0.9% sodium chloride for 2 minutes. One-third volume calcium chloride was added at a final concentration of 8.33 mM to induce clot formation that was automatically detected by the analyzer and reported in seconds from the time the CaCl₂ was applied.

Thrombelastography

Thrombelastography was carried out with a Haemoscope Computerized Thromboelastograph (CTEG) 5000. Disposable cups and pins were used according to the supplier's guidelines. A thrombin assay was performed using thrombin (10 U/ml final concentration) and CaCl₂ (7.77 mM final concentration). The parameters measured were the R-time, K-time, Angle (α), and Maximum Amplitude (MA). The R-time is the period of time of latency from the time that the sample was placed in the analyzer until the initial fibrin formation. The K-time is a measure of the speed to reach a certain level of clot strength. The Angle measures the rapidity of fibrin build-up and cross-linking. The MA is a direct function of the maximum dynamic properties of fibrin and platelet bonding via GPIIb/IIIa and represents the ultimate strength of the fibrin clot.

Platelet Agglutination by Ristocetin

Platelet agglutination by ristocetin was measured with a Lumni-Aggregometer (Chrono-Log Corporation). Platelet count was adjusted at 300K cells/with PPP and ristocetin was added to the suspension at a final concentration of 5 mg/ml. Aggregation/agglutination results can be measured as a percentage of maximum light transmittance as determined by PPP. Using AGGRO/LINK the slope(S) and amplitude (A) of the generated curve was computed. Platelet agglutination by ristocetin was also analyzed visually at 4 and at 16x magnification utilizing a Leitz Light Microscope (Wetzler, Germany).

Hypotonic Shock Response (HSR)

HSR was measured using a Chronolog SPA 2000 according to the standard procedure described in the instrument's instruction manual. HSR measures the platelet's ability to recover its normal volume from swelling when exposed to a hypotonic environment. Percent HSR, or % HSR (% recovery), is calculated and recorded automatically by the instrument.

Moisture Analysis

Residual moisture present in the lyophilized products was determined by the loss-on-drying (LOD) method using a Sartorius Moisture Analyzer (Edgewood, N.Y.). Sample preparation and testing took place in a controlled environment glove box that was continuously purged with dry nitrogen to keep the relative humidity near 0%. Samples of 1-2 grams were crushed and spread evenly over a pre-tarred aluminum weighing pan. The analyzer was set to automatic mode heating the sample to 100° C. with an infrared lamp and drying the sample until no further change in weight was detected. Data was reported in percent moisture (the ratio of the weights pre and post heating).

Numeric Recovery of Platelets After Freeze-Drying

Platelets from five different donors were stabilized and lyophilized at 500K cells/μl. Platelet counts of non-treated, and stabilized platelets were taken both pre-and post-lyophilization. About 76% of the original, pre lyophilization count is recovered post lyophilization in the non-treated platelet preparation. In contrast, 100% of the original count was recovered in all stabilized platelet preparations.

Effect of Freeze-Drying on Platelet Size Distribution and Morphology

Non-treated platelets underwent a substantial increase in platelet size after lyophilization and reconstitution with distilled water. Fresh platelets had a mean platelet volume (MPV) about 7.3 fL. Non-treated lyophilized platelets had a MPV about 8.9 fL. In contrast, stabilized lyophilized platelets displayed a size distribution almost identical to the size distribution of fresh platelets. Microscopic examination showed severe distortion of platelet morphology and formation of small aggregates in the non-treated lyophilized platelet suspension. In contrast, stabilized lyophilized platelets displayed a well-preserved, platelet morphology and no signs of aggregate formation.

Effect of Freeze-Drying on Cytosolic LDH Release

Release of cytosolic LDH is an indicator of membrane damage and cell lysis. It is important to sufficiently stabilize the platelets before freeze-drying to protect them from lysing during the freeze-drying/rehydration process. LDH release was measured in PPP's isolated from non-treated lyophilized-, and stabilized lyophilized platelets, with or without sucrose added prior to lyophilization. Data were compiled from five different donors and presented in Table 1. As can be seen from the table, the platelet stabilization procedure per se significantly reduced the amount of LDH release as a result of lyophilization. The addition of sucrose to PPP prior to lyophilization almost completely eliminates LDH release from stabilized platelets. Non-treated platelets released approximately 20 times more LDH compared to stabilized lyophilized platelets, which indicates severe membrane damage and/or cell lysis. Addition of sucrose prior to lyophilization did not protect non-treated platelets from membrane damage and/or cell lysis as determined by LDH release.

Effect of Freeze-Drying on Platelet Response to Ristocetin

Stabilized lyophilized platelets agglutinated in response to ristocetin. Agglutination was measured with a Lumni-Aggregometer and characterized by an amplitude, A=27±5%. In contrast, non-treated lyophilized platelets did not agglutinate in the presence of ristocetin (A=0). Data were compiled from five different donors and presented as mean values ±SE. Microscopic observation confirmed these results.

Effect of Freeze-Drying on Platelet Thromboplastic Function

The recalcification time test is used primarily to measure platelet thromboplastic function. Providing platelets have a normal thromboplastic function, the recalcification time of platelet-rich plasma (PRP) should be shorter compared to the recalcification time of platelet poor plasma (PPP).

Recalcification times of PRP containing non-treated platelets, PRP containing stabilized platelets and autologous PPP, were measured both pre- and post-lyophilization. The recalcification time of fresh non-treated PRP (248±16 sec) was significantly shorter than the recalcification time of fresh PPP (322±19 sec) indicating a normal platelet thromboplastic function. The recalcification time of fresh, stabilized PRP (185±4 sec) was significantly shorter than the recalcification time of fresh non-treated PRP (248±16 sec) indicating that the stabilization procedure stimulates the platelet thromboplastic function.

Lyophilization caused a significant decrease in the recalcification time for PPP and non-treated PRP. The recalcification time of fresh PPP was reduced by about 25% after lyophilization. Similarly, the recalcification time of fresh non-treated PRP is reduced by about 29% after lyophilization. In contrast, the post lyophilization recalcification time of stabilized PRP was not statistically different from the pre-lyophilization recalcification time (P=0.06).

Effect of Freeze-Drying on Clot Strength Measured by Thrombelastography

Thrombelastography analysis was performed on fresh PRP, fresh donor plasma (FDP), lyophilized PRP containing non-treated platelets, and lyophilized PRP containing stabilized platelets. MA of fresh plasma is only 7.5 mm. In contrast, MA of fresh PRP is substantially higher: 70.9 mm. Thus, MA clearly represents the platelet contribution to the clot strength. MA was reduced by approx. 59% after lyophilization in both non-treated and stabilized PRP. MA of stabilized, lyophilized PRP is slightly higher (30.0 mm) as compared to MA of non-treated, lyophilized PRP (26.5 mm). However, the difference may be statistically insignificant (P=0.60).

Effect of Freeze-Drying on Hypotonic Shock Response (HSR)

HSR of fresh platelets, non-treated lyophilized, and stabilized lyophilized platelets was measured. HSR scores of fresh platelets varied between 65-85%. Non-treated lyophilized platelets had completely lost their ability to recover the normal volume from swelling when exposed to a hypotonic environment. And HSR scores were approx. 0. In contrast, stabilized lyophilized platelets had preserved some of their original ability to control the volume under hypotonic conditions. HSR scores for stabilized lyophilized platelets varied between 10-25%. Data were collected from five different donors.

Moisture Content

Moisture content in the freeze-dried samples was below 2% as determined by the loss-on-drying (LOD) method.

Other embodiments and uses of the invention will be apparent to those skilled in the art from consideration of the specification and practice of the invention disclosed herein. All references cited herein, including all publications, U.S. and foreign patents and patent applications, are specifically and entirely incorporated by reference. It is intended that the specification and examples be considered exemplary only.

This invention has been described by reference to generic practices, and by description through specific examples. The examples are not intended to be limiting, alternatives, in terms of reagents, times and temperatures, will occur to those of skill in the art without the exercise of inventive faculty. Such alternatives remain within the scope of the invention disclosed herein, save for exclusion by express limitation in the claims set forth below.

TABLE 1
Data were compiled from five different donors
and presented as mean values ± SE.

| | LDH (U/L) in the supernatant |
|---|---|
| Product | of 500K cells/μl |
| Non-Treated Lyophilized Platelets | 1,068 ± 25 |
| Non-Treated Lyophilized | 1,060 ± 28 |
| Platelets + 150 mM sucrose | |
| Stabilized Lyophilized Platelets | 70 ± 15 |
| Stabilized Lyophilized | 65.5 ± 12 |
| Platelets + 60 mM Sucrose | |
| Stabilized Lyophilized | 50 ± 10 |
| Platelets + 150 mM Sucrose | |

1. A lyophilized preparation of blood platelets wherein, prior to lyophilization, said blood platelets are stabilized with glyceraldehyde, genipin, glyoxal, an analog thereof, or a combination thereof.

2. The preparation of claim 1, wherein said platelets are resuspended in at least one of autologous plasma, allogenic plasma, a high molecular weight polymer and combinations thereof prior to lyophilization.

3. The preparation of claim 2, wherein said high molecular weight polymer is at least one of a dextran, a hydroxyethyl starch, a modified gelatin, an albumin and combinations thereof.

4. The preparation of claim 2, wherein said autologous or allogenic plasma is a platelet poor plasma.

5. The preparation of claim 4, wherein said platelet poor plasma further comprises at least one of a sucrose, a trehalose, a glycine, a dymethyl sulfoxide and combinations thereof.

6. The preparation of claim 1, wherein said blood platelets are responsive to hypotonic stress.

7. The preparation of claim 1, which is substantially free of toxic chemicals.

8. The preparation of claim 1, wherein said platelets flexible.

9. The preparation of claim 1, wherein the glyceraldehyde analog is selected from the group consisting of dl-glyceraldehyde, dl-glyceraldehyde dimer, glyoxal, and combinations and mixtures thereof.

10. A reconstituted lyophilized platelet preparation, wherein subsequent to reconstitution, said platelets exhibit a size distribution and freedom from aggregation that is substantially indistinguishable from control platelets that have not been subject to lyophilization.

11. The preparation of claim 10, wherein said platelets are reconstituted in at least one of a pH adjusted matrix of autologous plasma, allogenic plasma and combinations thereof.

12. The preparation of claim 10, wherein said platelets are reconstituted in at least one of distilled, deionized, distilled-deionized, autoclaved, sterile saline, ultra pure pathogen free and combinations thereof.

13. The preparation of claim 10, wherein, upon reconstitution, the platelets release LDH in an amount corresponding to less than 100 U/L in a five hundred thousand cells/μl concentration.

14. The preparation of claim 10, wherein, upon reconstitution, the platelets aggregate in the presence of ristocetin.

15. The preparation of claim 10, wherein, upon reconstitution, the platelets exhibit a hypotonic shock response of approximately 10-25% or more.

16. A method of preparing lyophilized platelets, comprising stabilizing platelets from a donor in the presence of a glyceraldehyde analog at 30-40° C. for a period of 1-3 hours, followed by washing, suspension in a pH buffered protein matrix, and freeze drying the resuspended platelets.

17. The method of claim 16, further comprising stabilizing the platelets in at least one of glyceraldehyde, genipin, glyoxal, an analog thereof, or a combination thereof.

18. The method of claim 17, further comprising resuspending the platelets in a glyceraldehyde-dimer.

19. The method of claim 16, further comprising resuspending the platelets in at least one of autologous plasma, allogenic plasma, a high molecular weight polymer and combinations thereof prior to lyophilization.

20. The method of claim 16, further comprising reconstituting the stabilized lyophilized platelets.
